(19) United States
(12) Patent Application Publication
(10) Pub. No.: US 2022/0187270 A1
(43) Pub. Date: Jun. 16, 2022

(54) Method for Evaluating Carbon Source Quality of Water Body, Apparatus, Device and Readable Storage Medium

(71) Applicants: Tongji University, Shanghai (CN); Nanjing University, Nanjing (CN)

(72) Inventors: Yinguang Chen, Shanghai (CN); Xiong Zheng, Shanghai (CN); Hongqiang Ren, Shanghai (CN); Bing Wu, Shanghai (CN)

(21) Appl. No.: 17/250,222

(22) PCT Filed: Nov. 26, 2020
(86) PCT No.: PCT/CN2020/131622

§ 371 Date: Dec. 17, 2020

(30) Foreign Application Priority Data
May 26, 2020 (CN) 2020 10457795.4

(51) Int. Cl. G01N 33/18; C02F 1/00

(52) U.S. Cl. CPC G01N 33/1806; C02F 2101/105; C02F 2209/08; C02F 1/001

(57) ABSTRACT

The invention relates to the technical field of environmental protection, in particular to a method for evaluating carbon source quality of a water body, an apparatus, a device and a readable storage medium. The invention provides a method for evaluating carbon source quality of a water body, including: acquiring COD and BOD₅ of a first water body, wherein the first water body is a water body obtained after a water body to be measured is subjected to filtration treatment; acquiring an energy matter content in microbial cells in a second water body, wherein the second water body is a water body obtained after the first water body is subjected to anaerobic-aerobic treatment; and determining the carbon source quality of the water body to be measured according to a ratio of COD to BOD₅ in the first water body and the energy matter content in the microbial cells in the second water body. The method for evaluating carbon source quality of the water body provided by the invention can effectively solve the existing problems of partial evaluation and poor pertinence of sewage biodegradability, implements accurate evaluation of the sewage carbon source on the biological nitrogen and phosphorus removal process, has the advantages of wide adaptability, accurate evaluation and the like, and has good industrialization prospects.

### BACKGROUND

### Technical Field

The invention relates to the technical field of environmental protection, in particular to a method for evaluating carbon source quality of a water body, an apparatus, a device and a readable storage medium.

### Related Art

The current situation in the prevention and control of water body pollution in China is still severe, and nitrogen and phosphorus pollution is still an important reason for eutrophication of water bodies. The high contents of nitrogen and phosphorus are generally high in urban and rural sewage treatment in China. In order to deal with this problem, China has built and operated nearly 4000 urban sewage treatment plants, of which more than 90% adopt the activated sludge process. In the process of treating nitrogen and phosphorus in sewage by using activated sludge microorganisms, both the removal of phosphorus and the denitrification and nitrogen removal require the use of carbon sources. Therefore, the quality of the sewage carbon source, that is, whether it is conducive to biological nitrogen and phosphorus removal, is an important factor that is directly related to the urban sewage treatment effect in China, so as to ensure the quality of the ecological environment of the water body.

### SUMMARY

In view of the above-mentioned defects in the prior art, an object of the invention is to provide a method for evaluating carbon source quality of a water body, an apparatus, a device and a readable storage medium to solve the problems in the prior art.

In order to realize the above object and other related objects, one aspect of the invention provides a method for evaluating carbon source quality of a water body, including:

1) acquiring COD and BOD₅ of a first water body, wherein the first water body is a water body obtained after a water body to be measured is subjected to filtration treatment;

2) acquiring an energy matter content in microbial cells in a second water body, wherein the second water body is a water body obtained after the first water body is subjected to anaerobic-aerobic treatment; and

3) determining the carbon source quality of the water body to be measured according to a ratio of COD to BOD₅ in the first water body and the energy matter content in the microbial cells in the second water body.

In some implementations of the invention, in step 1), when solid phase matter trapped by the filtration treatment is ≥30 mg/L, the first water body is a water body obtained after the water body to be measured is subjected to filtration treatment and anaerobic treatment.

In some implementations of the invention, in step 1), a pore size of a filter medium in the filtration treatment is 0.4 to 0.5 μm.

In some implementations of the invention, in step 1), the filtration treatment is filtration treatment with a filter membrane.

In some implementations of the invention, in step 1), an oxidation-reduction potential of the anaerobic treatment is −100 mV to −150 mV, and a reaction time is 18 to 24 hours.

In some implementations of the invention, in step 2), a total time of the anaerobic-aerobic treatment is 8 to 12 hours, a treatment time of an anaerobic stage is ≥1.5 hours, a sludge concentration is 2500 to 3500 mg/L, a C/N/P ratio is (90 to 110):(4.5 to 5.5):(0.9 to 1.1), a temperature is 20 to 30° C., a pH is 6.5 to 7.5, a dissolved oxygen content in an aerobic stage is ≥2 mg/L, and a dissolved oxygen content in the anaerobic stage is ≤0.5 mg/L.

In some implementations of the invention, in step 2), energy matter is selected from polyhydroxyvaleric acid (PHV).

In some implementations of the invention, the carbon source quality specifically refers to whether the water body is a carbon source suitable for biological nitrogen and phosphorus removal.

In some implementations of the invention, when the ratio of COD to BOD₅ is higher, the water body is considered to have better carbon source quality.

In some implementations of the invention, when the energy matter content is higher, the water body is considered to have better carbon source quality.

Another aspect of the invention provides a computer-readable storage medium, having a computer program stored thereon. When the program is executed by a processor, the method for evaluating carbon source quality of a water body is implemented.

Another aspect of the invention provides an apparatus, including: a processor and a memory. The memory is configured to store a computer program, and the processor is configured to execute the computer program stored on the memory such that the apparatus executes the method for evaluating carbon source quality of a water body.

Another aspect of the invention provides a device. The device is capable of including: a COD and BOD₅ acquisition module, configured to acquire COD and BOD₅ of a first water body, wherein the first water body is a water body obtained after a water body to be measured is subjected to filtration treatment;

a PHV content acquisition module, configured to acquire a PHV content in microbial cells in a second water body, wherein the second water body is a water body obtained after the first water body is subjected to anaerobic-aerobic treatment; and

a water body carbon source quality determination module, configured to determine the carbon source quality of the water body to be measured according to a ratio of COD to BOD₅ in the first water body and the energy matter content in the microbial cells in the second water body.

### DETAILED DESCRIPTION

Inventors of the invention found through lots of research that carbon source quality of a water body can be evaluated more accurately through a ratio of COD to BOD₅ and an energy matter content, thereby providing a novel method for evaluating carbon source quality of a water body, an apparatus, a device and a readable storage medium. The invention is completed on this basis.

A first aspect of the invention provides a method for evaluating carbon source quality of a water body, including:

1) COD (chemical oxygen demand) and BOD₅ (biological oxygen demand) of a first water body are acquired. The first water body is a water body obtained after a water body to be measured is subjected to filtration treatment;

2) An energy matter content in microbial cells in a second water body is acquired. The second water body is a water body obtained after the first water body is subjected to anaerobic-aerobic treatment.

3) The carbon source quality of the water body to be measured is determined according to a ratio of COD to BOD₅ in the first water body and the energy matter content in the microbial cells in the second water body.

In the method for evaluating carbon source quality of the water body, the water body to be measured may generally be urban domestic sewage and/or industrial wastewater, etc. The water body to be measured may generally include components such as organic matter and nutritive salts. For example, a concentration of the organic matter COD may be ≤1 mg/L, 1 to 5 mg/L, 5 to 10 mg/L, 10 to 20 mg/L, 20 to 40 mg/L, 40 to 60 mg/L, 60 to 100 mg/L, 100 to 200 mg/L, 200 to 300 mg/L, 300 to 500 mg/L or 500 to 1000 mg/L. For the water body to be measured, the organic matter COD may generally be in the above range, and if it exceeds the above range, the water body may generally be diluted. A concentration of the nutritive salts in the water body to be measured is generally not particularly limited. Preferably, a C/N/P ratio in the water body to be measured may meet (90 to 110):(4.5 to 5.5):(0.9 to 1.1).

The method for evaluating carbon source quality of the water body provided by the invention may include: acquiring COD and BOD₅ of a first water body, wherein the first water body is a water body obtained after a water body to be measured is subjected to filtration treatment. The filtration treatment generally refers to a treatment method in which a fluid is trapped by a suitable filter medium, thereby separating solid particles in the fluid from the liquid. In the filtration treatment, a pore size of the filter medium is 0.4 to 0.5 μm, 0.4 to 0.42 μm, 0.42 to 0.44 μm, 0.44 to 0.46 μm, 0.46 to 0.48 μm or 0.48 to 0.5 μm. Generally speaking, before COD and BOD₅, the water body needs to be subjected to the filtration treatment with a filter medium with a suitable pore size to ensure the accuracy of the measurement results. Those skilled in the art may select a suitable method to perform the filtration treatment on the water body to be measured. For example, the filtration treatment may be filtration treatment with a filter membrane and the like.

In the method for evaluating carbon source quality of the water body provided by the invention, when the content of solid phase matter in the water body to be measured is too high, it is generally necessary to consider dissolved organic matter and particulate organic matter respectively. For example, when the solid phase matter trapped by the filtration treatment is ≥20 mg/L, ≥25 mg/L, ≥30 mg/L, ≥35 mg/L, or ≥40 mg/L, it is generally necessary to perform filtration treatment on the water body to be measured and then perform anaerobic treatment on the water body subjected to the filtration treatment, and then, the COD and BOD₅ of the water body are acquired. Specifically, suspended solids also contain organic matter, but microorganisms in a sewage treatment system cannot directly use the particulate organic matter to synthesize polyhydroxyalkanoic acid (PHA), so the anaerobic treatment may be used to convert particulate organic matter into dissolved organic matter for the synthesis of PHA, and finally it is determined whether the sewage can be used to synthesize a higher amount of PHV and whether the nitrogen and phosphorus removal effect of sewage is good. Generally speaking, in the water body obtained after the water body subjected to the filtration treatment is subjected to the anaerobic treatment, the concentration of the dissolved organic matter will greatly increase, and they may be used in the subsequent sewage treatment process to synthesize the energy matter PHA by microorganisms. The anaerobic treatment generally refers to a treatment method in which nutritional conditions and environmental conditions required by anaerobic microorganisms are formed in the water body under anaerobic conditions and organic matter in the water body is biochemically degraded through the metabolism of anaerobic bacteria and facultative bacteria. Those skilled in the art may select a suitable method to perform anaerobic treatment on the water body to be measured. For example, in the method for performing anaerobic treatment on the water body to be measured, an oxidation-reduction potential may be −100 mV to −150 mV, −100 mV to −110 mV, −110 mV to −120 mV, −120 mV to −130 mV, −130 mV to −140 mV, or −140 mV to −150 mV. For another example, a reaction time may be 18 to 24 hours, 18 to 20 hours, 20 to 22 hours, or 2 to 24 hours. For another example, a temperature may be 20 to 30° C., 20 to 25° C., or 25 to 30° C. For another example, a pH may be 6.5 to 7.5, 6.5 to 6.7, 6.7 to 6.9, 6.9 to 7, 7 to 7.1, 7.1 to 7.3, or 7.3 to 7.5. For another example, a C/N/P ratio may be (90 to 110):(4.5 to 5.5):(0.9 to 1.1). For another example, a sludge concentration may be 2500 to 3500 mg/L, 2500 to 2700 mg/L, 2700 to 2900 mg/L, 2900 to 3100 mg/L, 3100 to 3300 mg/L, or 3300 to 3500 mg/L. For another example, in the method for performing anaerobic treatment on the water body to be measured, a dissolved oxygen content may be ≤0.5 mg/L.

The method for evaluating carbon source quality of the water body provided by the invention may further include: acquiring an energy matter content in microbial cells in a second water body, wherein the second water body is a water body obtained after the first water body is subjected to anaerobic-aerobic treatment. Generally speaking, in the water body obtained after the first water body is subjected to the anaerobic-aerobic treatment, the particulate organic matter is significantly decreased and the dissolved organic matter is increased, which is beneficial to the subsequent utilization of microorganisms. The anaerobic-aerobic treatment generally refers to a treatment method in which a water body is subjected to anaerobic treatment and aerobic treatment sequentially, and the aerobic treatment generally refers to a treatment method in which aerobic microorganisms (including facultative microorganisms) performs biological metabolism in the presence of oxygen to degrade organic matter in the water body. Those skilled in the art may select a suitable method to perform anaerobic-aerobic treatment on the first water body. For example, in the anaerobic-aerobic treatment on the first water body, a total time of the anaerobic-aerobic treatment is 8 to 12 hours. A treatment time of an anaerobic stage is ≥1.5 hours. For another example, in the method for performing anaerobic treatment on the water body to be measured, an oxidation-reduction potential may be −100 mV to −150 mV, −100 mV to −110 mV, −110 mV to −120 mV, −120 mV to −130 mV, −130 mV to −140 mV, or −140 mV to −150 mV. For another example, a temperature may be 20 to 30° C., 20 to 25° C., or 25 to 30° C. For another example, a pH may be 6.5 to 7.5, 6.5 to 6.7, 6.7 to 6.9, 6.9 to 7, 7 to 7.1, 7.1 to 7.3, or 7.3 to 7.5. For another example, a C/N/P ratio may be (90 to 110):(4.5 to 5.5):(0.9 to 1.1). For another example, a sludge concentration may be 2500 to 3500 mg/L, 2500 to 2700 mg/L, 2700 to 2900 mg/L, 2900 to 3100 mg/L, 3100 to 3300 mg/L, or 3300 to 3500 mg/L. For another example, in the anaerobic-aerobic treatment on the first water body, a dissolved oxygen content of an aerobic stage may be ≥2 mg/L, and a dissolved oxygen content of the anaerobic stage is ≤0.5 mg/L.

In the method for evaluating carbon source quality of the water body provided by the invention, a suitable method for acquiring the energy matter content in the microbial cells in the water body should be known to those skilled in the art. For example, the method may include: centrifuging and drying the second water body, and measuring the content of energy matter in the solid phase matter.

The method for evaluating carbon source quality of the water body provided by the invention may further include: determining the carbon source quality of the water body to be measured according to a ratio of COD to BOD₅ in the first water body and the energy matter content in the microbial cells in the second water body. The carbon source quality specifically refers to whether the water body is a carbon source suitable for biological nitrogen and phosphorus removal, that is, if the water body is considered to have higher carbon source quality, it is considered that the water body is relatively more suitable for the biological nitrogen and phosphorus removal technique, and conversely, if the water body is considered to have worse carbon source quality, it is considered that the water body is relatively not suitable for the biological nitrogen and phosphorus removal technique. The biological nitrogen and phosphorus removal generally refers to a treatment method in which biological treatment is used to remove the nutrients nitrogen and phosphorus in the water body. In the biological nitrogen and phosphorus removal technique, the key to the biological phosphorus removal of the water body is phosphorus-accumulating bacteria. The currently recognized biological phosphorus removal mechanism believes that the biological phosphorus removal needs to undergo two stages respectively: an anaerobic stage and an aerobic stage. In the anaerobic stage, the phosphorus-accumulating bacteria hydrolyze polyphosphates, absorb the sewage carbon source, and synthesize energy matter. This process is accompanied by the release of phosphates (that is, the anaerobic phosphorus release process). In the aerobic stage, the phosphorus-accumulating bacteria use the energy matter to metabolize and re-synthesize polyphosphates in the absence of an external carbon source (that is, the aerobic excess phosphorus uptake process), and finally, surplus sludge containing the phosphorus-accumulating bacteria is separated and discharged to achieve the goal of removing phosphorus from the sewage. The process of removing nitrogen in the water body is mainly a denitrification and nitrogen removal process, which refers to a biochemical process in which under anaerobic conditions, denitrifying microorganisms in the activated sludge uses the carbon source in the microbial cells as an electron donor to reduce nitrogen in nitrates (NO₃⁻) into nitrogen gas (N₂) through a series of metabolic intermediates (that is, nitrite NO₂⁻, nitric oxide NO and nitrous oxide N₂O). In the biological nitrogen and phosphorus removal process, the carbon source of the water body to be treated is not directly used by the microorganisms of the activated sludge, but is transformed into a carbon source in the cells and further metabolized by the microorganisms to achieve the biological phosphorus removal and denitrification process. The biological nitrogen and phosphorus removal may generally include, but not limited to, A²O, Bardenpho, UCT, Phoredox, SBR and other techniques. Generally speaking, if the water body has high carbon source quality, that is, the water body is suitable for biological nitrogen and phosphorus removal, the microorganisms can better complete the nitrogen and phosphorus removal process, so that the treated effluent water has better water quality and can better meet the relevant national discharge standards. The ratio of COD to BOD₅ in the first water body may reflect the effect of degradation on the water body by aerobic organisms. When the B/C ratio (that is, the ratio of BOD₅ to COD) is higher, it indicates a better effect of degradation on the sewage by the aerobic organisms, that is, the water body to be measured is generally considered to have better carbon source quality. Conversely, if the B/C ratio is lower, the water body to be measured is considered to have worse carbon source quality. However, the B/C ratio alone cannot accurately reflect the carbon source quality of the water body to be measured in deed, because the B/C ratio is used to evaluate the biodegradability of the sewage carbon source based on degradation of organic matter in the sewage by the aerobic microorganisms, and the aerobic microorganisms are not all microorganisms having biological nitrogen and phosphorus removal functions. For example, denitrifying microorganisms are anaerobic microorganisms. The inventors of the invention unexpectedly discovered that the energy matter content in the microbial cells in the second water body, preferably the content of polyhydroxyalkanoate (PHA) in the microbial cells in the second water body, more preferably the content of polyhydroxyvalerate (PHV) in the microbial cells in the second water body, has a close relationship with the carbon source quality of the water body to be measured, and can more effectively reflect the carbon source quality of the water body to be measured. Generally speaking, if the PHV content in the microbial cells in the second water body is higher, the water body to be measured is considered to have better carbon source quality, and conversely, if the PHV content is lower, the water body to be measured is considered to have worse carbon source quality. The combination of the ratio of COD to BOD₅ in the first water body and the energy matter content in the microbial cells in the second water body can more accurately indicate the degree to which the water body is suitable for biological nitrogen and phosphorus removal. In a specific embodiment of the invention, when the B/C ratio is greater than 0.1 and the PHV content is greater than or equal to 200 mg/kg dry cell weight, the water body to be measured is considered to be a high-quality carbon source suitable for biological nitrogen and phosphorus removal. When the B/C ratio is greater than 0.1 and the PHV content is 50-200 mg/kg dry sludge, the water body to be measured is considered to be a common carbon source that can be used for biological nitrogen and phosphorus removal. When the B/C ratio is greater than 0.1 and the PHV content is less than 50 mg/kg dry sludge, the water body to be measured is considered to be a carbon source not suitable for biological nitrogen and phosphorus removal. For a water body of which B/C is less than 0.1, the water body is generally not treated by biological methods, so it is basically impossible to determine whether the water body is a high-quality carbon source for nitrogen and phosphorus removal.

A second aspect of the invention provides a computer-readable storage medium, having a computer program stored thereon. When the program is executed by a processor, the method for evaluating carbon source quality of the water body provided by the first aspect of the invention is implemented.

A third aspect of the invention provides an apparatus, including: a processor and a memory. The memory is configured to store a computer program, and the processor is configured to execute the computer program stored on the memory such that the apparatus executes the method for evaluating carbon source quality of the water body provided by the first aspect of the invention.

A fourth aspect of the invention provides a device. The device is capable of including:

a COD and BOD₅ acquisition module, configured to acquire COD and BOD₅ of a first water body, wherein the first water body is a water body obtained after a water body to be measured is subjected to filtration treatment;

a PHV content acquisition module, configured to acquire a PHV content in microbial cells in a second water body, wherein the second water body is a water body obtained after the first water body is subjected to anaerobic-aerobic treatment; and

a water body carbon source quality determination module, configured to determine the carbon source quality of the water body to be measured according to a ratio of COD to BOD₅ in the first water body and the energy matter content in the microbial cells in the second water body.

In the invention, for the operating principle of modules in the above device, reference may be made to the method for evaluating carbon source quality of the water body provided by the first aspect of the invention, and details will not be repeated here.

The method for evaluating carbon source quality of the water body provided by the invention can effectively solve the existing problems of partial evaluation and poor pertinence of sewage biodegradability, implements accurate evaluation of the sewage carbon source on the biological nitrogen and phosphorus removal process, has the advantages of wide adaptability, accurate evaluation and the like, and has good industrialization prospects.

The implementations of the invention are described below through specific examples. Those skilled in the art can easily understand the other advantages and effects of the invention from the content disclosed in the specification. The invention can also be implemented or applied through other different specific implementations, and various details in the specification can also be modified or changed based on different viewpoints and applications without departing from the spirit of the invention.

It should be noted that the technological apparatuses or devices not specifically noted in the following embodiments all adopt conventional apparatuses or devices in the art.

In addition, it should be understood that the one or more method steps mentioned in the invention do not exclude that there may be other method steps before and after the combined step or that other method steps may also be inserted between these steps explicitly mentioned, unless otherwise specified. It should also be understood that the combined connection relationship between one or more apparatuses/devices mentioned in the invention does not exclude that there may be other apparatuses/devices before and after the combined apparatus/device or that other apparatuses/devices may also be inserted between these two apparatuses/devices explicitly mentioned, unless otherwise specified. Moreover, unless otherwise specified, the number of each method step is only a convenient tool to identify each method step, not to limit the arrangement order of the method steps or limit the implementable scope of the invention, and changes or adjustments of the relative relationship between them shall also be regarded as the implementable scope of the invention without substantial changes in the technical content.

### Embodiment 1

(1) Sewage to be evaluated (mainly containing organic matter (COD), nutrients (N, P and the like) and the like) was filtered through a 0.45 μm filter membrane to respectively obtain a filtrate and a suspended solid.

(2) The concentration of the suspended solid was measured to be 25 mg/L, which was less than the standard of 30 mg/L. The chemical oxygen demand (COD) and the biological oxygen demand (BOD₅) of the filtrate were directly measured (for the specific method, reference was made to Water and Wastewater Monitoring Analysis Method (Fourth Edition)) to be 400 mg/L and 160 mg/L respectively.

(3) The obtained BOD₅/COD ratio 0.4 was used as the B/C ratio of the sewage, that is, B/C=0.4, indicating that the sewage to be evaluated was a carbon source suitable for biological nitrogen and phosphorus removal.

(4) The sewage to be evaluated in step 2 was connected into a stably acclimated A²O technique biological nitrogen and phosphorus removal system, and subjected to conventional anaerobic and aerobic treatment. Then, 100 mL of a microbial mixed solution in the nitrogen and phosphorus removal system was taken out.

(5) After microorganisms obtained in step 4 were centrifuged and subjected to vacuum freeze drying, the content of polyhydroxyvaleric acid (PHV) in the obtained solid was measured by gas chromatography to be 230 mg/kg respectively.

(6) According to the established evaluation standard, the sewage carbon source to be evaluated was a high-quality carbon source for biological nitrogen and phosphorus removal.

(7) The sewage to be evaluated (in which the concentration of total nitrogen TN was 35 mg/L and the concentration of total phosphorus TP was 6 mg/L) was directly connected into a conventional stably-operating A²O technique biological nitrogen and phosphorus removal system to verify the carbon source quality. Specific parameters of the A²O technique biological nitrogen and phosphorus removal system were as follows: a hydraulic retention time was 18 hours (wherein a retention time in the anaerobic zone was 1.5 hours, a retention time in the anoxic zone was 5 hours, and a retention time in the aerobic zone was 11.5 hours), a sludge concentration was 3000 mg/L, and a dissolved oxygen content in an aerobic stage was ≥2 mg/L. After the measurement, a TN removal rate of the system reached 80%, and a TP removal rate reached 98%, proving that the sewage carbon source to be evaluated was a high-quality carbon source for biological nitrogen and phosphorus removal.

### Embodiment 2

(1) Sewage to be evaluated (mainly containing organic matter (COD), nutrients (N, P and the like) and the like) was filtered through a 0.45 μm filter membrane to respectively obtain a filtrate and a suspended solid.

(2) The concentration of the suspended solid was measured to be 25 mg/L, which was less than the standard of 30 mg/L. The chemical oxygen demand (COD) and the biological oxygen demand (BOD₅) of the filtrate were directly measured (for the specific method, reference was made to Water and Wastewater Monitoring Analysis Method (Fourth Edition)) to be 300 mg/L and 210 mg/L respectively.

(3) The obtained BOD₅/COD ratio 0.7 was used as the B/C ratio of the sewage, that is, B/C=0.7, indicating that the sewage to be evaluated was a carbon source suitable for biological nitrogen and phosphorus removal.

(4) The sewage to be evaluated in step 2 was connected into a stably acclimated A²O technique biological nitrogen and phosphorus removal system, and subjected to conventional anaerobic and aerobic treatment. Then, 100 mL of a microbial mixed solution in the nitrogen and phosphorus removal system was taken out.

(5) After microorganisms obtained in step 4 were centrifuged and subjected to vacuum freeze drying, the content of polyhydroxyvaleric acid (PHV) in the obtained solid was measured by gas chromatography to be 40 mg/kg respectively.

(6) According to the established evaluation standard, the sewage carbon source to be evaluated was a carbon source not suitable for biological nitrogen and phosphorus removal.

(7) The sewage to be evaluated (in which the concentration of total nitrogen TN was 30 mg/L and the concentration of total phosphorus TP was 6 mg/L) was directly connected into a conventional stably-operating A²O technique biological nitrogen and phosphorus removal system (the same as Embodiment 1) to verify the carbon source quality. After the measurement, a TN removal rate of the system was 55%, and a TP removal rate was 40%, proving that the sewage carbon source to be evaluated was a carbon source not suitable for biological nitrogen and phosphorus removal.

### Embodiment 3

(1) Sewage to be evaluated (mainly containing organic matter (COD), nutrients (N, P and the like) and the like) was filtered through a 0.45 μm filter membrane to respectively obtain a filtrate and a suspended solid.

(2) The concentration of the suspended solid was measured to be 100 mg/L, which was greater than the standard of 30 mg/L. The sewage to be evaluated was subjected to anaerobic treatment. An oxidation-reduction potential of the anaerobic treatment was −150 mV, and a reaction time was 24 hours. The chemical oxygen demand (COD) and the biological oxygen demand (BOD₅) of the filtrate of the formed first water body were measured (for the specific method, reference was made to Water and Wastewater Monitoring Analysis Method (Fourth Edition)) to be 600 mg/L and 180 mg/L respectively.

(3) The obtained BOD₅/COD ratio 0.3 was used as the B/C ratio of the sewage, that is, B/C=0.3, indicating that the sewage to be evaluated was a carbon source suitable for biological nitrogen and phosphorus removal.

(4) The sewage to be evaluated in step 2 was connected into a stably acclimated A²O technique biological nitrogen and phosphorus removal system, and subjected to conventional anaerobic and aerobic treatment. Then, 100 mL of a microbial mixed solution in the nitrogen and phosphorus removal system was taken out.

(5) After microorganisms obtained in step 4 were centrifuged and subjected to vacuum freeze drying, the content of polyhydroxyvaleric acid (PHV) in the obtained solid was measured by gas chromatography to be 200 mg/kg respectively.

(6) According to the established evaluation standard, the sewage carbon source to be evaluated was a high-quality carbon source for biological nitrogen and phosphorus removal.

(7) The sewage to be evaluated (in which the concentration of total nitrogen TN was 40 mg/L and the concentration of total phosphorus TP was 8 mg/L) was directly connected into a conventional stably-operating A²O technique biological nitrogen and phosphorus removal system (the same as Embodiment 1) to verify the carbon source quality. After the measurement, a TN removal rate of the system reached 80%, and a TP removal rate reached 95%, proving that the sewage carbon source to be evaluated was a high-quality carbon source for biological nitrogen and phosphorus removal.

### Embodiment 4

(1) Sewage to be evaluated (mainly containing organic matter (COD), nutrients (N, P and the like) and the like) was filtered through a 0.45 μm filter membrane to respectively obtain a filtrate and a suspended solid.

(2) The concentration of the suspended solid was measured to be 100 mg/L, which was greater than the standard of 30 mg/L. The sewage to be evaluated was subjected to anaerobic treatment. An oxidation-reduction potential of the anaerobic treatment was −150 mV, and a reaction time was 24 hours. The chemical oxygen demand (COD) and the biological oxygen demand (BOD₅) of the filtrate of the formed first water body were measured (for the specific method, reference was made to Water and Wastewater Monitoring Analysis Method (Fourth Edition)) to be 800 mg/L and 480 mg/L respectively.

(3) The obtained BOD₅/COD ratio 0.6 was used as the B/C ratio of the sewage, that is, B/C=0.6, indicating that the sewage to be evaluated was a carbon source suitable for biological nitrogen and phosphorus removal.

(4) The sewage to be evaluated in step 2 was connected into a stably acclimated A²O technique biological nitrogen and phosphorus removal system, and subjected to conventional anaerobic and aerobic treatment. Then, 100 mL of a microbial mixed solution in the nitrogen and phosphorus removal system was taken out.

(5) After microorganisms obtained in step 4 were centrifuged and subjected to vacuum freeze drying, the content of polyhydroxyvaleric acid (PHV) in the obtained solid was measured by gas chromatography to be 30 mg/kg respectively.

(6) According to the established evaluation standard, the sewage carbon source to be evaluated was a carbon source not suitable for biological nitrogen and phosphorus removal.

(7) The sewage to be evaluated (in which the concentration of total nitrogen TN was 60 mg/L and the concentration of total phosphorus TP was 15 mg/L) was directly connected into a conventional stably-operating A²O technique biological nitrogen and phosphorus removal system (the same as Embodiment 1) to verify the carbon source quality. After the measurement, a TN removal rate of the system was 50%, and a TP removal rate was 30%, proving that the sewage carbon source to be evaluated was a carbon source not suitable for biological nitrogen and phosphorus removal.

### Embodiment 5

(1) Sewage to be evaluated (mainly containing organic matter (COD), nutrients (N, P and the like) and the like) was filtered through a 0.45 μm filter membrane to respectively obtain a filtrate and a suspended solid.

(2) The concentration of the suspended solid was measured to be 30 mg/L, which was equal to the standard of 30 mg/L. The sewage to be evaluated was subjected to anaerobic treatment. An oxidation-reduction potential of the anaerobic treatment was −150 mV, and a reaction time was 24 hours. The chemical oxygen demand (COD) and the biological oxygen demand (BOD₅) of the filtrate of the formed first water body were measured (for the specific method, reference was made to Water and Wastewater Monitoring Analysis Method (Fourth Edition)) to be 400 mg/L and 200 mg/L respectively.

(3) The obtained BOD₅/COD ratio 0.5 was used as the B/C ratio of the sewage, that is, B/C=0.5, indicating that the sewage to be evaluated was a carbon source suitable for biological nitrogen and phosphorus removal.

(4) The sewage to be evaluated in step 2 was connected into a stably acclimated A²O technique biological nitrogen and phosphorus removal system, and subjected to conventional anaerobic and aerobic treatment. Then, 100 mL of a microbial mixed solution in the nitrogen and phosphorus removal system was taken out.

(5) After microorganisms obtained in step 4 were centrifuged and subjected to vacuum freeze drying, the content of polyhydroxyvaleric acid (PHV) in the obtained solid was measured by gas chromatography to be 250 mg/kg respectively.

(6) According to the established evaluation standard, the sewage carbon source to be evaluated was a high-quality carbon source for biological nitrogen and phosphorus removal.

(7) The sewage to be evaluated (in which the concentration of total nitrogen TN was 50 mg/L and the concentration of total phosphorus TP was 10 mg/L) was directly connected into a conventional stably-operating A²O technique biological nitrogen and phosphorus removal system (the same as Embodiment 1) to verify the carbon source quality. After the measurement, a TN removal rate of the system reached 85%, and a TP removal rate reached 99%, proving that the sewage carbon source to be evaluated was a high-quality carbon source for biological nitrogen and phosphorus removal.

### Embodiment 6

(1) Sewage to be evaluated (mainly containing organic matter (COD), nutrients (N, P and the like) and the like) was filtered through a 0.45 μm filter membrane to respectively obtain a filtrate and a suspended solid.

(2) The concentration of the suspended solid was measured to be 30 mg/L, which was equal to the standard of 30 mg/L. The sewage to be evaluated was subjected to anaerobic treatment. An oxidation-reduction potential of the anaerobic treatment was −150 mV, and a reaction time was 24 hours. The chemical oxygen demand (COD) and the biological oxygen demand (BOD₅) of the filtrate of the formed first water body were measured (for the specific method, reference was made to Water and Wastewater Monitoring Analysis Method (Fourth Edition)) to be 400 mg/L and 200 mg/L respectively.

(3) The obtained BOD₅/COD ratio 0.5 was used as the B/C ratio of the sewage, that is, B/C=0.5, indicating that the sewage to be evaluated was a carbon source suitable for biological nitrogen and phosphorus removal.

(4) The sewage to be evaluated in step 2 was connected into a stably acclimated A²O technique biological nitrogen and phosphorus removal system, and subjected to conventional anaerobic and aerobic treatment. Then, 100 mL of a microbial mixed solution in the nitrogen and phosphorus removal system was taken out.

(5) After microorganisms obtained in step 4 were centrifuged and subjected to vacuum freeze drying, the content of polyhydroxyvaleric acid (PHV) in the obtained solid was measured by gas chromatography to be 45 mg/kg respectively.

(6) According to the established evaluation standard, the sewage carbon source to be evaluated was a carbon source not suitable for biological nitrogen and phosphorus removal.

(7) The sewage to be evaluated (in which the concentration of total nitrogen TN was 50 mg/L and the concentration of total phosphorus TP was 10 mg/L) was directly connected into a conventional stably-operating A²O technique biological nitrogen and phosphorus removal system (the same as Embodiment 1) to verify the carbon source quality. After the measurement, a TN removal rate of the system was 60%, and a TP removal rate was 50%, proving that the sewage carbon source to be evaluated was a carbon source not suitable for biological nitrogen and phosphorus removal.

In summary, the invention effectively overcomes various defects in the prior art and has high industrial value in use.

The above embodiments only exemplarily illustrate the principles and effects of the invention, but are not used to limit the invention. Anyone familiar with the art can modify or change the above embodiments without departing from the spirit and scope of the invention. Therefore, all equivalent modifications or changes made by those of ordinary skill in the art without departing from the spirit and technical ideas disclosed in the invention should still be covered by the claims of the invention.

1. A method for evaluating carbon source quality of a water body, comprising:
1) acquiring COD and BOD₅ of a first water body, wherein the first water body is a water body obtained after a water body to be measured is subjected to filtration treatment;
2) acquiring an energy matter content in microbial cells in a second water body, wherein the second water body is a water body obtained after the first water body is subjected to anaerobic-aerobic treatment; and
3) determining the carbon source quality of the water body to be measured according to a ratio of COD to BOD₅ in the first water body and the energy matter content in the microbial cells in the second water body.

2. The method for evaluating carbon source quality of a water body according to claim 1, wherein in step 1), when solid phase matter trapped by the filtration treatment is 30 mg/L, the first water body is a water body obtained after the water body to be measured is subjected to filtration treatment and anaerobic treatment.

3. The method for evaluating carbon source quality of a water body according to claim 1, wherein in step 1), a pore size of a filter medium in the filtration treatment is 0.4 to 0.5 μm;
and/or, in step 1), the filtration treatment is filtration treatment with a filter membrane.

4. The method for evaluating carbon source quality of a water body according to claim 2, wherein in step 1), an oxidation-reduction potential of the anaerobic treatment is −100 mV to −150 mV, and a reaction time is 18 to 24 hours.

5. The method for evaluating carbon source quality of a water body according to claim 1, wherein in step 2), a total time of the anaerobic-aerobic treatment is 8 to 12 hours, a treatment time of an anaerobic stage is ≥1.5 hours, a sludge concentration is 2500 to 3500 mg/L, a C/N/P ratio is (90 to 110):(4.5 to 5.5):(0.9 to 1.1), a temperature is 20 to 30° C., a pH is 6.5 to 7.5, a dissolved oxygen content in an aerobic stage is ≥2 mg/L, and a dissolved oxygen content in the anaerobic stage is ≤0.5 mg/L.

6. The method for evaluating carbon source quality of a water body according to claim 1, wherein in step 2), energy matter is selected from polyhydroxyvaleric acid.

7. The method for evaluating carbon source quality of a water body according to claim 1, wherein the carbon source quality specifically refers to whether the water body is a carbon source suitable for biological nitrogen and phosphorus removal;
and/or, when the ratio of COD to BOD₅ is higher, the water body is considered to have better carbon source quality;
and/or, when the energy matter content is higher, the water body is considered to have better carbon source quality.

8. A computer-readable storage medium, having a computer program stored thereon, wherein when the program is executed by a processor, the method for evaluating carbon source quality of a water body according to claim 1 is implemented.

9. An apparatus, comprising: a processor and a memory, wherein the memory is configured to store a computer program, and the processor is configured to execute the computer program stored on the memory such that the apparatus executes the method for evaluating carbon source quality of a water body according to claim 1.

10. A device, comprising:
a COD and BOD₅ acquisition module, configured to acquire COD and BOD₅ of a first water body, wherein the first water body is a water body obtained after a water body to be measured is subjected to filtration treatment;
a PHV content acquisition module, configured to acquire an energy matter content in microbial cells in a second water body, wherein the second water body is a water body obtained after the first water body is subjected to anaerobic-aerobic treatment; and
a water body carbon source quality determination module, configured to determine the carbon source quality of the water body to be measured according to a ratio of COD to BOD₅ in the first water body and the energy matter content in the microbial cells in the second water body.
